(19) United States
(12) Patent Application Publication
(10) Pub. No.: US 2021/0077668 A1
(43) Pub. Date: Mar. 18, 2021

(54) Highly Adherent Polymers for Orthopedic Device Coatings

(71) Applicants: Clemson University Research Foundation, Clemson, SC (US); MUSC Foundation for Research Development, Charleston, SC (US)

(72) Inventors: Alexey A. Vertegel, Clemson, SC (US); Igor A. Luzinov, Clemson, SC (US); Christopher E. Gross, Charleston, SC (US); Dmitry Gil, Clemson, SC (US); Nikolay Borodinov, Clemson, SC (US)

(21) Appl. No.: 16/959,562

(22) PCT Filed: Jan. 4, 2019
(86) PCT No.: PCT/US19/12346

§ 371 Date: Jul. 1, 2020

Related U.S. Application Data
(60) Provisional application No. 62/613,830, filed on Jan. 5, 2018.

(51) Int. Cl. A61L 31/10; A61L 31/16; C08F 220/28; A61B 17/84

(52) U.S. Cl. CPC A61L 31/10; A61L 31/16; C08F 220/286; A61B 17/848; A61B 2017/00889; A61L 2300/404; A61L 2420/06; A61L 2430/24; A61L 2430/02

(57) ABSTRACT

Provided is an orthopedic implant comprising a surface with a coating on the surface wherein the coating comprises a copolymer defined by Formula I: A_{w}-Bₓ-C_{y}-D_{z} wherein: A comprises an epoxy group; B comprises a hydrophobic group; C is an optional cross-linker; D of Formula I comprises a hydrophilic group; w is at least 0.1 to no more than 0.9 with the proviso that at least one of x or z is not zero; x is up to 0.9; y is up to 0.3; and z is up to 0.9.

### CROSS-REFERENCE TO RELATED APPLICATIONS

The present invention claims priority to pending U.S. Provisional Patent Application No. 62/613,830 filed Jan. 5, 2018 which is incorporated herein by reference.

### FIELD OF THE INVENTION

The present invention is related to a coating for medical implants, particularly orthopedic devices, wherein the coating has superior adhesion to the medical implant and the coating is capable of slow release of antimicrobials thereby mitigating infection rates.

### BACKGROUND

Orthopedic implants are available for many applications including joint replacements, bone repair fixtures and the like without limit thereto. For the purposes of the instant invention there are two general classes of orthopedic implants with one class being exclusively internal and typically permanent, such as a hip joint or shoulder joint, and the other being partially internal and temporary, such as Kirshner wires. All orthopedic implants are capable of housing bacteria and other undesirable materials and therefore infection due to an orthopedic implant is a particularly severe problem.

Pin site infections arise from the use of percutaneous pinning techniques such as those employed in skeletal traction, percutaneous fracture pinning, external fixation for fracture stabilization or complex deformation reconstruction. These sites are niduses for infection because the skin barrier is disrupted which allows for bacteria to enter at the junction of the skin and pin. After external fixation the rate of pin site infections is usually high and, in some circumstances and techniques, approaches 100%. Following pin site infection the pin may become loose which causes increased pain and the integrity of the fracture fixation may be compromised resulting in structural deformity and inferior clinical results. The excessive pain is also related to increased narcotic usage which is a critical secondary consideration. While many of the pin site infections are treatable with adequate wound care and oral antibiotics, osteomyelitis and deep soft tissue infections may occur with evidence of up to 4% of the cases escalating to a requirement for a more complex care plan. Due to the morbidity and costs associated with its sequelae, strategies to reduce pin site infections are vital.

Patients with Kirshner wires are particularly vulnerable to infection. Kirshner wires, often referred to as K-wires in the art, are sharpened pins, typically of stainless steel or titanium, which are inserted into the body for holding or positioning bones or for immobilization of a joint. K-wires typically extend outside the body thereby creating an air interface where the orthopedic device and surgical site meet and provide a potential site for infection. Therefore, even if the surgical procedure is accomplished without introduction of infection, the surgical site is subject to post-surgical infection. The infection rate following a K-wire procedure ranges from 11% to 100% depending on the procedure, facility and other parameters. Infection can result in sepsis, osteomyelitis and mortality if not treated properly. It has been estimated that the economic burden of infections following K-wire procedures will exceed one billion dollars by 2020 in the U.S. alone.

Staphylococcal infections account for about 80% of the infections observed after K-pin procedures. Mitigating this infection alone would have a significant impact on the number and severity of post-surgical infections observed in K-wire recipients. There have been many efforts associated with the formation of coatings, particularly on K-wires, to form a surface which is less susceptible to absorption of bacteria or capable of being impregnated with antimicrobials. Unfortunately, it is difficult to achieve adequate adhesion to metals and the coatings typically either delaminate or, if they survive simulated implant, the anti-microbial release is uncontrolled.

There has been a significant need for mitigating of the infection rate associated with orthopedic implants, in general, and K-wires specifically. Provided herein is a coating which is particularly suitable for orthopedic inserts wherein the coating has sufficient adhesion to survive implant and the coating provides for a slow release of antimicrobials.

### SUMMARY OF THE INVENTION

The present invention is related to a coating for orthopedic implants wherein the coating has superior adhesion and provides for a slow release of antimicrobials.

More specifically, the present invention is related to a coating for orthopedic implants, and improved orthopedic implants comprising a coating, wherein the coating has sufficient adhesion to the orthopedic implant to survive surgical implant and the coating comprises antimicrobials which are released at a controlled rate.

A particular feature of the invention is the ability to adjust the release rate of a specific antimicrobial by alteration of the coating.

These and other embodiments, as will be realized, are provided in an orthopedic implant comprising a surface with a coating on the surface wherein the coating comprises a copolymer defined by Formula I:

A_{w}-Bₓ-C_{y}-D_{z} Formula I

wherein:
A comprises an epoxy group or alkoxy silyl group;
B comprises a hydrophobic group;
C is an optional cross-linker;
D comprises a hydrophilic group;
w is at least 0.1 to no more than 0.9 with the proviso that at least one of x or z is not zero;
x is up to 0.9;
y is up to 0.3; and
z is up to 0.9.

Yet another embodiment is provided in a copolymer defined by Formula I:

A_{w}-Bₓ-C_{y}-D_{z} Formula I
wherein:
A comprises an epoxy or alkoxy silyl group and
B comprises a hydrophobic group;
C is an optional cross-linker;
D of Formula I comprises a hydrophilic group;
w is at least 0.1 to no more than 0.9 with the proviso that at least one of x or z is not zero;
x is up to 0.9;
y is up to 0.3; and
z is up to 0.9.

### BRIEF DESCRIPTION OF DRAWINGS

FIG. 1 is a graphical representation of an embodiment of the invention.

FIG. 2 is a graphical representation of an embodiment of the invention.

FIG. 3 is a graphical representation of an embodiment of the invention.

FIG. 4 is a graphical representation of an embodiment of the invention.

### DESCRIPTION

The present invention is related to improved orthopedic devices comprising a coating with superior adhesion to the orthopedic device wherein the coating is strongly adherent to the metal surface, remains intact upon application of shear and bending forces typically associated with an orthopedic surgery and maintains structural integrity during placement of the implant. A particular feature is the coating is capable of slow release of a loaded drug. Particularly preferred are drugs selected from the group consisting of anti-inflammatory drugs, antimicrobial drugs, anticancer drugs, antioxidant drugs or growth factor drugs with any other compatible drug being suitable for use.

The present invention is specific to a polymeric coating comprising a copolymer formed by the polymerization of a monomer comprising an epoxy terminal group with a mixture of monomers comprising hydrophobic groups and hydrophilic groups. The epoxy termination crosslinks with hydroxyl groups on the metal thereby providing adhesion of the polymeric matrix to the metal. The hydrophobic and hydrophilic components of the co-polymer allow for control of antimicrobial release rate and drug affinity.

The coating on the implant surface will be fabricated using a grafting method supplemented with cross-linking of the coating. The grafting method includes reaction of functionalized polymers with complimentary functional groups located on the substrate surface. One of the advantages of the grafting method is that the reaction does not require elaborate synthetic procedures. The synthesis and modification are sequential and therefore the conditions of the synthesis are not complicated by the presence of the substrate being coated which increases flexibility with regards to the materials which can be easily coated.

The surface modification process is preferably accomplished by dissolving previously prepared copolymers in a solvent, preferably water. The copolymer is then deposited as a film on the surface being modified by any suitable technique preferably selected from dip-coating, spray-coating or drop-casting. The coating thickness can be easily controlled by copolymer concentration and other processing parameters such as residence time in solution or deposition amount.

While not limited thereto, the polymer is preferable formed by radical polymerization which is well known to those of skill in the art and further detail thereof is not necessary.

A particularly preferred polymer is defined by Formula I:

A_{w}-Bₓ-C_{y}-D_{z} Formula I

wherein the formula represents a copolymer of monomers chosen from A, B, C and D, as will be more fully described herein, and the subscripts represent the mole fraction of each monomer in the copolymer and therefore the sum of w, x, y and z is unity. The copolymer can be a random copolymer wherein A, B, C and D are randomly distributed, a block co-polymer comprising discrete blocks of each monomer, periodic copolymers wherein the monomers are arranged in a repeating sequence, statistical copolymers wherein the sequence follows a statistical rule or combinations thereof throughout the polymer chain.

Component A of Formula I comprises an epoxy group and is represented in the copolymer by the formula:

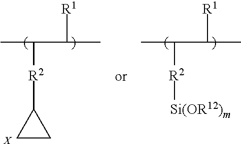

wherein:
X is O or N and preferably O;
R¹ is a hydrogen or alkyl of up to 4 carbons;
R² is a linking group preferably selected from alkyl of 2 to 5 carbons and —C(O)—O—CH₂—;
R¹² is an alkyl of up to 10 carbons which may be substituted.

The epoxy or alkoxy silyl group reacts with the metal surface and provides for strong adhesion of the coating to the metal surface. Epoxy groups also provide excellent storage properties and can remain stable for as much as six months in water. Under acid or base conditions the ring opens and is reactive with any nucleophilic group such as the hydroxyl groups on the surface of a metal.

A particularly preferred component A is a glycidyl methacrylate (PGMA) moiety represented in the polymer by:

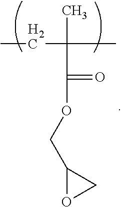

Another particularly preferred group for component A is an alkoxy silyl. Alkoxy silyls provide strong adhesion of the coating to the metal surface and ensures structural integrity of the coating during the application of shear and bending forces. A particularly preferred alkoxy silyl group is 3-(trimethoxysilyl) methacrylate represented by the following structure:

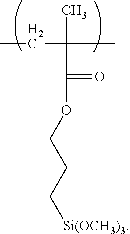

Component B of Formula I comprises a hydrophobic group represented in the copolymer by the formula:

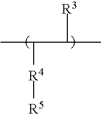

wherein:
R³ is a hydrogen or alkyl of up to 4 carbons;
R⁴ is a linking group preferably selected from alkyl of 2 to 5 carbons and —C(O)—O—CH₂—; and
R⁵ is an alkyl of 6 to 100 carbons and more preferably 10 to 100 carbons.

A particularly preferred component B is lauryl methacrylate (LMA) represented by the polymerized monomer:

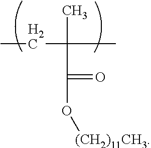

Component C of Formula I is an optional cross-linker, and preferably a UV cross-linker, capable of crosslinking with other groups within the copolymer thereby providing additional adhesion or polymeric strength. Component C of Formula I is represented in the polymer by the formula:

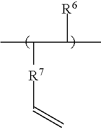

wherein:
R⁶ is a hydrogen or alkyl of up to 4 carbons; and
R⁷ is a linking group preferably selected from alkyl of 2 to 5 carbons and —C(O)—O—CH₂—.

A particularly preferred component C is allyl methacrylate (AMA) represented by the polymerized monomer:

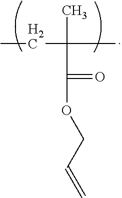

Component D of Formula I comprises a hydrophilic group providing water solubility, swellability, protein repellency and a matrix. Compound D is represented in the polymer by the formula:

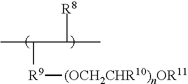

wherein:
R⁸ is a hydrogen or alkyl of up to 4 carbons;
R⁹ is a linking group selected from alkyl of 2 to 5 carbons and —C(O)—;
R¹⁹ is a hydrogen or alkyl of up to 4 carbons;
R¹¹ is a hydrogen or alkyl of up to 4 carbons; and
n is at least 2 to no more than 25.

Poly (oligo(ethylene glycol) methyl ether methacrylate) (POEGMA) as component D has particularly desirable protein/cell repellency properties and the ability to compatibilize materials with water. The reactive methacrylate moiety is capable of undergoing polymerization while quite long poly ethylene glycol moieties provide water compatibility to the copolymer. The poly ethylene glycol moieties are known to have low toxicity and do not trigger immune system responses. A particularly preferred component D is polymerized ethylene glycol methacrylate (OEGMA) represented by the polymerized monomer:

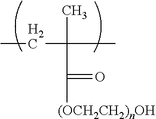

wherein n is preferably an integer sufficient to achieve a molecular weight of 300 to 2000 and preferably 800 to 1200. Particularly preferred component D is OEGMA wherein n is selected from the integers 2-9, or combinations thereof, wherein the hydrophilicity increases with increasing n.

In Formula I the subscripts are defined by molar ratio such that after polymerization the copolymer is represented as one mole. In Formula I, w is at least 0.1 to no more than 0.9 with at least one of x or z is not zero. Below about 0.1 mole fraction the number of epoxy groups is insufficient to form an adhesive bond to the metal. Above a mole fraction of about 0.9 there is insufficient hydrophobic or hydrophilic moieties to absorb a sufficient amount of antimicrobial to be beneficial. More preferably, w is at least 0.1 to no more than 0.85, even more preferably at least 0.1 to no more than 0.73, even more preferably at least 0.1 to no more than 0.6 and most preferably at least 0.1 to no more than 0.3.

In Formula I, x is up to 0.9 and preferably at least 0.01 up to 0.9. The molar ratio of component B is determined based on the degree of hydrophobicity required to achieve the release rate. A higher portion of the hydrophobic moiety will slow water absorption and therefore decrease the release rate of hydrophilic antimicrobials. If the hydrophobic moiety is too high the antimicrobials cannot be absorbed in the copolymer. More preferably, x is at least 0.2 to no more than 0.8 and even more preferably at least 0.5 to no more than 0.7.

In Formula I, y is up to 0.3 and preferably 0.001 up to 0.3. The optional cross-linker provides additional intra-polymer cross-linking which increases the mechanical robustness of the polymer. If the degree of cross-linking is excessive the microbacterial is unable to be absorbed and released from the copolymer matrix. More preferably, y is no more than 0.05. The optional cross-linker allows for secondary cross-linking if necessary such as by UV activation.

In Formula I, z is up to 0.9 and preferably 0.01 up to 0.9. The molar ratio of component D is determined based on the degree of hydrophilicity required to achieve the release rate. A higher portion of the hydrophilic moiety will increase water absorption and therefore increase the release rate of hydrophilic antimicrobials. More preferably, z is at least 0.1 to no more than 0.5 and even more preferably at least 0.1 to no more than 0.3.

The copolymer is formed on the surface of the metal to form an adequate coating which preferably does not exceed about 0.1 wt % of the mass of the K-wire or about 100 μM thickness. Above a thickness of about 100 μm the coating becomes less robust and deterioration is observed upon insertion through a Septa™ used to simulate surgical insertion. More preferably, the thickness of the coating is about 0.1 to 5 μm and even more preferably about 0.5 to 1 μm.

The surface of the metal can be used as is or treated to increase the number of surface hydroxyl groups thereby increasing the bonding sites available for reaction with the epoxy or alkoxy silyl group. The surface may be on an exclusively internal orthopedic device, such as a replacement joint, or a partially external orthopedic device such as a Kirshner wire. The surface is not particularly limited herein with the proviso that the surface have cross-linkable groups on the surface such as hydroxyl groups. Titanium, stainless steel and ceramic surfaces are particularly preferred.

### EXPERIMENTAL

### Example 1

Control K-wires were coated with monolaurin (ML), a natural antimicrobial agent active against S. aureus. Sample K-wires were coated with a copolymer formed from 20 mole percent glycidyl methacrylate, 60 mole percent ethylene glycol methacrylate and 20 mole percent ethylene glycol methacrylate available as OEGMA 950 from Sigma Aldrich. Some of the K-wires were pulled through Septa™, to simulate surgical insertion, and the antimicrobial activity was measured. Each example was replicated nine times. The results are provided in Table 1:

TABLE 1

| | Maximum concentration of bacteria inactivated in the | | |
|---|---|---|---|
| Concentration of | presence of 1 cm pieced of K-wires | | |
| ML | No Polymer, | No Polymer after | With Polymer after |
| mg/ml | no Septa | Septa | Septa |
| 1 | ~6 × 10⁶ | 0 | ~6 × 10⁶ |
| 3 | ~7 × 10⁶ | 0 | ~8 × 10⁷ |
| 5 | ~6 × 10⁶ | 0 | ~7 × 10⁸ |
| 10 | ~8 × 10⁶ | 0 | ~2 × 10⁹ |

Based on the results of Table 1 subsequent experiments reported herein utilized a coating solution comprising 10 wt % ML and 5 wt % polymer unless otherwise stated.

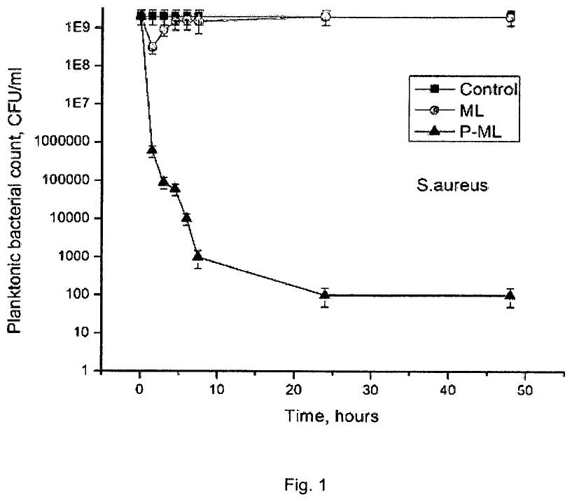

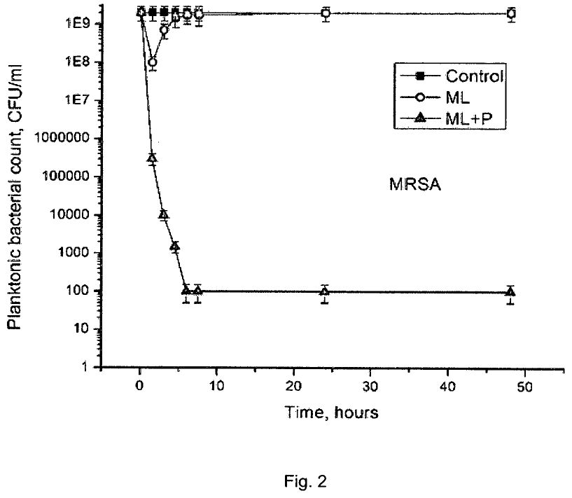

Additional tests were done wherein bacterial count was monitored versus time for different bacteria. In FIG. 1 the bacteria count was measured for S. aureus versus time. The K-wire alone as a control was ineffective as expected. Samples coated with ML only demonstrated an immediate decrease in bacteria due to essentially immediate release of ML, however, the activity was not sustained. The sample prepared with ML in polymer demonstrated a steady decrease in bacteria to a plateau of about 100 CFU/ml which represents negligible bacterial concentration. The test was repeated with Methicillin-Resistant Staphylococcus Aureus (MRSA) as the bacteria with results similar to those observed for S. aureus as illustrated graphically in FIG. 2.

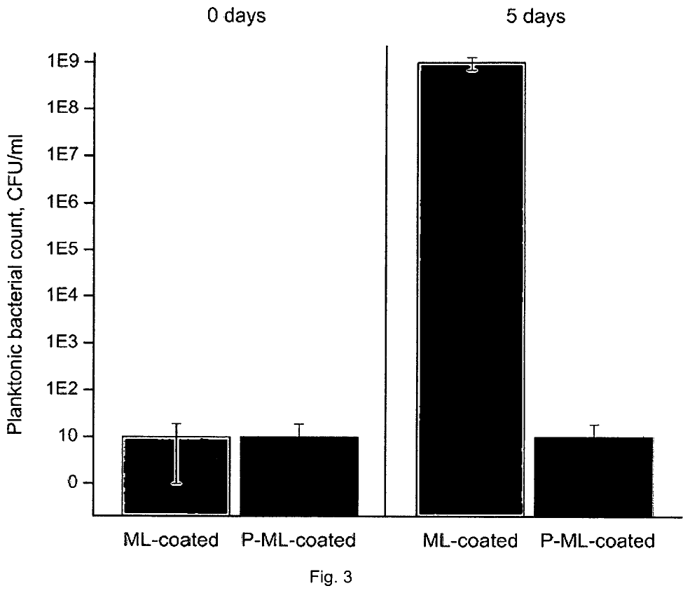

The storage stability of monolaurin in the polymer layer on a K-wire was compared to monolaurin on a K-wire without the polymer layer. The antibacterial activity of the monolaurin coating after 5 days at 50° C., corresponding to about 45 days at room temperature, was measured against 10⁵ CFU of planktonic S. aureus. The results are presented graphically in FIG. 3. The wires were not passed through a Septa™ for these test. The results demonstrate an improved storage stability for the inventive examples.

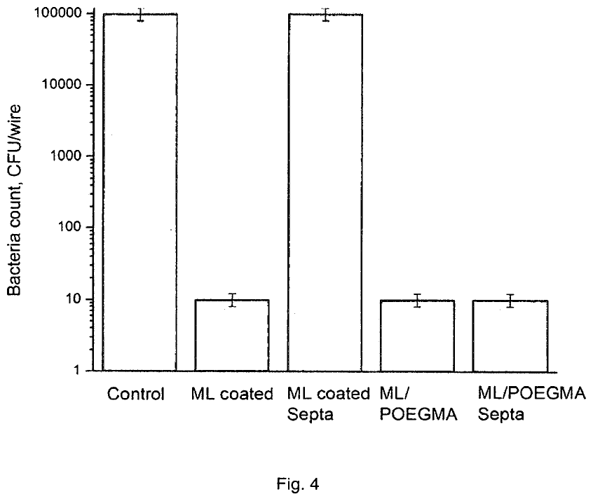

Effectiveness of the inventive examples against biofilms was determined. Samples were prepared including a control K-wire (Control), a K-wire coated with ML only but not passed through a Septa™ (ML Coated), a sample treated with ML and passed through a Septa™ (ML Coated Septa), and inventive examples comprising ML with polymer, using OEGMA as the hydrophilic moiety (ML/POEGMA). Some of the inventive samples were passed through a Septa™ (ML/POEGMA Septa). The samples were all incubated in S. aureus for 48 hours. The results are provided graphically in FIG. 4. As realized from the results presented in FIG. 4 the ML coated sample demonstrates effective resistance unless passed through a Septa™ suggesting the ML coating is removed or otherwise rendered ineffective. The inventive examples are not negatively impacted by passing through the Septa™ which indicates a robust coating on the surface.

### Example 2

An 0.062″ stainless steel Kirscher wire was dip-coated with Copolymer A comprising a 15/66/19 molar ratio of GMA, OEGMA and LMA. Copolymer A was deposited from a methylether ketone (MEK) solvent with a 2.5 wt % polymer concentration. The coating was thermally treated at 80° C. for 5 hours to crosslink the reactive groups. The polymer thickness was about 900 nm as determined by atomic force microscopy. The polymer layer was loaded with monolaurin, as a water insoluble antimicrobial, and vancomycin, as a water-soluble anti-microbial to test release rate. The antimicrobials were added by introducing the coated polymer to an MEK solution comprising the antimicrobial. Wires dip-coated in polyactide (PLA) from acetone solution were used as controls. The inventive coating remained virtually intact after pulling the coated wire through a Septa™ cap. In the case of the PLA coated wire, further studies using scanning electron microscopy showed evidence of deterioration of the polymer layer whereas the inventive coating showed no signs of mechanical or structural deterioration.

### Example 3

The performance of Copolymer A was compared to poly(lactide-co-glycolide) PLGA) with monolaurin as a model antimicrobial additive. S. aureus biofilms were grown by statically incubating a S. Aureus suspension with 1 cm K-wire pieces for 48 hours. Monolaurin incorporated into both coatings, and a coating of monolaurin with no polymer layer were all efficient in preventing biofilm formation. However, after pulling the wires through silicon rubber Septa™ caps both the monolaurin, with no polymer coating, and the PLGA based coating lost the antimicrobial activity while the sample utilizing Copolymer A remained efficient against biofilm formation.

### Example 4

K-wires were prepared as in Example 3 with vancomycin as the antimicrobial. The K-wires were drilled into a mechanically equivalent femoral bone construct available from Sawbones USA as Model #3414. The wires were then removed from the drill, rinsed by deionized water and cut into pieces. The pieces that were exposed to the bone were placed into test tubes containing 10⁷ colony forming units (CFU) if S. aureus. Only the K-wires with a Copolymer A coating containing vancomycin showed antimicrobial activity. Control samples comprising a K-wires with a Copolymer A coating but no vancomycin where not efficient confirming that Copolymer A does not provide antimicrobial activity.

### Example 5

The cytotoxicity of CoPolymer A was evaluated. A coating was applied as discussed above and the effects on cell proliferation were evaluated in a pilot cell culture experiment with murine 7F2 osteoblasts (ATCC® CRL-12557) in a protocol adapted from open literature. Osteoblasts were passaged after reaching confluency and aliquots containing about 40,000 cells were transferred into a sterile 24-well plate containing uncoated wires, as a control, Copolymer A as an inventive example and PGMA homopolymer. The samples were incubated in the presence of the cells for 2, 4 and 8 days at 37° C. in 5% W CO2. Following the incubation the wires were removed from the wells and osteoblasts were exposed to 5 mg/ml MTT reagent (3-[4,5-dimethylthiazol-2yl]-2,5-diphenyl-tetrazolium bromide) for 4 hours followed by dissolution in dimethyl sulfoxide and measurement of optical density at 570 nm. Six replicates were performed for each of the time periods. Neither the inventive sample nor the PGMA coated wires had an effect on cell proliferation rate. The OD₅₇₀, which corresponds to the number of living cells, was not significantly different for coated and uncoated wires at different time points.

The invention has been described with reference to the preferred embodiments without limit thereto. Additional embodiments and improvements may be realized which are not specifically set forth herein but which are within the scope of the invention as more specifically set forth in the claims appended hereto.

1. An orthopedic device comprising:
a surface;
a coating on said surface wherein said coating comprises a copolymer defined by Formula I:
A_{w}-Bₓ-C_{y}-D_{z} Formula I
wherein:
A comprises an epoxy or alkoxy silyl group and B comprises a hydrophobic group;
C is an optional cross-linker;
D of Formula I comprises a hydrophilic group;
w is at least 0.1 to no more than 0.9 with the proviso that at least one of x or z is not zero;
x is up to 0.9;
y is up to 0.3; and
z is up to 0.9.

2. The orthopedic device of claim 1 wherein said A is represented in the copolymer by the formula: 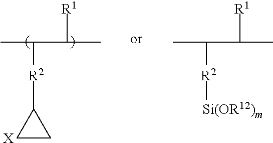
wherein:
X is O or N;
R¹ is a hydrogen or alkyl of up to 4 carbons;
R² is a linking group; and
R¹² is an alkyl of up to 10 carbons which may be substituted.

3. The orthopedic device of claim 2 wherein said X is O.

4. The orthopedic device of claim 2 wherein said R² is selected from alkyl of 2 to 5 carbons and —C(O)—O—CH₂—.

5. The orthopedic device of claim 2 wherein said A is polymerized glycidyl methacrylate or polymerized 3-(trimethoxysilyl) methacrylate.

6. The orthopedic device of claim 1 wherein said B is represented in the copolymer by the formula: 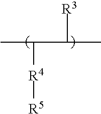
wherein:
R³ is a hydrogen or alkyl of up to 4 carbons;
R⁴ is a linking group; and
R⁵ is an alkyl of 10 to 100 carbons.

7. The orthopedic device of claim 6 wherein R⁴ is selected from alkyl of 2 to 5 carbons and —C(O)—O—CH₂—.

8. The orthopedic device of claim 6 wherein said B is polymerized lauryl methacrylate.

9. The orthopedic device of claim 1 wherein said C is represented in the polymer by the formula: 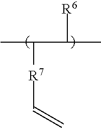
wherein:
R⁶ is a hydrogen or alkyl of up to 4 carbons; and
R⁷ is a linking group.

10. The orthopedic device of claim 9 wherein said R⁷ selected from alkyl of 2 to 5 carbons and —C(O)—O—CH₂—.

11. The orthopedic device of claim 9 wherein said C is polymerized allyl methacrylate.

12. The orthopedic device of claim 1 wherein said D is represented in the polymer by the formula: 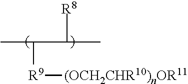
wherein:
R⁸ is a hydrogen or alkyl of up to 4 carbons;
R⁹ is a linking group;
R¹⁹ is a hydrogen or alkyl of up to 4 carbons;
R¹¹ is a hydrogen or alkyl of up to 4 carbons; and
n is at least 6 to no more than 25.

13. The orthopedic device of claim 12 wherein R⁹ is selected from alkyl of 2 to 5 carbons and —C(O)—.

14. The orthopedic device of claim 13 wherein said n is selected from the integers 2-9.

15. The orthopedic device of claim 1 wherein said w is at least 0.1 to no more than 0.3.

16. The orthopedic device of claim 1 wherein said x is at least 0.2 to no more than 0.8.

17. The orthopedic device of claim 16 wherein said x is at least 0.5 to no more than 0.7.

18. The orthopedic device of claim 1 wherein said y is no more than 0.05.

19. The orthopedic device of claim 1 wherein said z is at least 0.1 to no more than 0.5.

20. The orthopedic device of claim 19 wherein said z is at least 0.1 to no more than 0.3.

21. The orthopedic device of claim 1 wherein said surface comprises a material selected from titanium, stainless steel and ceramic.

22. The orthopedic device of claim 1 wherein said surface is on an internal device or a device which extends external to the patient.

23. The orthopedic device of claim 2 wherein said device is a Kirshner wire.

24. The orthopedic device of claim 1 wherein said Formula I is selected from a random copolymer, a block co-polymer, a periodic copolymer, a statistical copolymer and combinations thereof.

24. The orthopedic device of claim 1 wherein said coating is no more than 100 μm thick.

25. The orthopedic device of claim 24 wherein said coating is at least 0.1 to 5 μm thick.

26. The orthopedic device of claim 25 wherein said coating is at least 0.5 to no more than 1 μm thick.

27. The orthopedic device of claim 1 further comprising a drug incorporated into said coating.

28. A copolymer defined by Formula I:
A_{w}-Bₓ-C_{y}-D_{z} Formula I
wherein:
A comprises an epoxy or alkoxy silyl group and
B comprises a hydrophobic group;
C is an optional cross-linker;
D of Formula I comprises a hydrophilic group;
w is at least 0.1 to no more than 0.9 with the proviso that at least one of x or z is not zero;
x is up to 0.9;
y is up to 0.3; and
z is up to 0.9.

29. The copolymer of claim 28 wherein said A is represented in the copolymer by the formula: 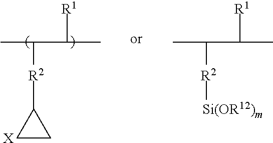
wherein:
X is O or N;
R¹ is a hydrogen or alkyl of up to 4 carbons;
R² is a linking group; and
R¹² is an alkyl of up to 10 carbons which may be substituted.

30. The copolymer of claim 29 wherein said X is O.

31. The copolymer of claim 29 wherein said R² is selected from alkyl of 2 to 5 carbons and —C(O)—O—CH₂—.

32. The copolymer of claim 29 wherein said A is polymerized glycidyl methacrylate or polymerized 3-(trimethoxysilyl) methacrylate.

33. The copolymer of claim 28 wherein said B is represented in the copolymer by the formula: 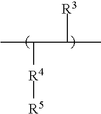
wherein:
R³ is a hydrogen or alkyl of up to 4 carbons;
R⁴ is a linking group; and
R⁵ is an alkyl of 10 to 100 carbons.

34. The copolymer of claim 33 wherein R⁴ is selected from alkyl of 2 to 5 carbons and —C(O)—O—CH₂—.

35. The copolymer of claim 33 wherein said B is polymerized lauryl methacrylate.

36. The copolymer of claim 28 wherein said C is represented in the polymer by the formula: 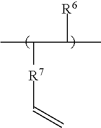
wherein:
R⁶ is a hydrogen or alkyl of up to 4 carbons; and
R⁷ is a linking group.

37. The copolymer of claim 36 wherein said R⁷ selected from alkyl of 2 to 5 carbons and —C(O)—O—CH₂—.

38. The copolymer of claim 36 wherein said C is polymerized allyl methacrylate.

39. The copolymer of claim 28 wherein said D is represented in the polymer by the formula: 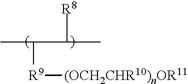
wherein:
R⁸ is a hydrogen or alkyl of up to 4 carbons;
R⁹ is a linking group;
R¹⁹ is a hydrogen or alkyl of up to 4 carbons;
R¹¹ is a hydrogen or alkyl of up to 4 carbons; and
n is at least 6 to no more than 25.

40. The copolymer of claim 39 wherein R⁹ is selected from alkyl of 2 to 5 carbons and —C(O)—.

41. The copolymer of claim 40 wherein said n is selected from the integers 2-9.

42. The copolymer of claim 28 wherein said w is at least 0.1 to no more than 0.3.

43. The copolymer of claim 28 wherein said x is at least 0.2 to no more than 0.8.

44. The copolymer of claim 43 wherein said x is at least 0.5 to no more than 0.7.

45. The copolymer of claim 28 wherein said y is no more than 0.05.

46. The copolymer of claim 28 wherein said z is at least 0.1 to no more than 0.5.

47. The copolymer of claim 46 wherein said z is at least 0.1 to no more than 0.3.

48. The copolymer of claim 28 wherein said Formula I is selected from a random copolymer, a block co-polymer, a periodic copolymer, a statistical copolymer and combinations thereof.

49. The copolymer of claim 29 further comprising a drug incorporated into said copolymer.
